(19) United States
(12) Patent Application Publication
(10) Pub. No.: US 2003/0219840 A1
(43) Pub. Date: Nov. 27, 2003

(54) Method of analyzing proenzyme forms of prostate specific antigen in serum to improve prostate cancer detection

(76) Inventors: Stephen D. Mikolajczyk, San Diego, CA (US); Harry G. Rittenhouse, Del Mar, CA (US)

Correspondence Address: PATENT LEGAL DEPARTMENT/A-42-C, BECKMAN COULTER, INC., 4300 N. HARBOR BOULEVARD, BOX 3100, FULLERTON, CA, 92834-3100, US

(21) Appl. No.: 10/154,715

(22) Filed: May 24, 2002

(51) Int. Cl. G01N 33/574

(52) U.S. Cl. 435/7.23

(57) ABSTRACT

Assays for detecting and determining the presence of prostate cancer is provided. The assays are capable of detecting prostate cancer in the population of men with significantly higher ratio of free PSA to total PSA. The assays are also capable of detecting prostate cancer in the population of men with low amount of total PSA, i.e., in the range of 2 to 4 ng/ml. In accordance with one embodiment of the present invention, the assay includes the steps of (a) determining the amount of total PSA contained in a biological sample from the patient, (b) determining the amount of free PSA in the sample; and calculating the ratio of the free PSA to the total PSA, (c) determining the amount of pPSA in the sample, and (d) correlating the amount of pPSA contained in the sample to the presence of prostate cancer in the patient by comparing the amount of pPSA to a predetermined value established with control samples of known cancer and benign disease diagnosis, based on both the level of total PSA and the % free PSA.

### FIELD OF THE INVENTION

The present invention relates generally to the detection and identification of proteins, as well as various forms and subunits of proteins, which have the potential utility as diagnostic markers. In particular, the present invention relates to the detection of inactive precursor forms of prostate specific antigens in serum, and methods for improved detection of prostate cancer.

### BACKGROUND OF THE INVENTION

The measurement of serum prostate specific antigen (PSA) is widely used for the screening and early detection of prostate cancer (1-3). Serum PSA that is measurable by current clinical immunoassays exists primarily as either the free “non-complexed” form (free PSA), or as a complex with α-₁lantichymotrypsin (ACT) (4-5). The ratio of free to total PSA in serum has been demonstrated to significantly improve the discrimination of PCa from BPH, with higher ratios correlating with a lower risk of prostate cancer (6-7).

The biological mechanism for the variable levels of free PSA in serum is unknown. The serum PSA that has become complexed is likely to be relatively homogeneous since this represents enzymatically active, intact PSA. The PSA released from the PSA-ACT complex in prostate cancer (PCa) and benign prostate hyperplasia (BPH) serum was found to be indistinguishable from seminal plasma PSA, which confirms this assumption (8). It follows that free PSA may offer better biochemical insight, and that a characterization of the molecular forms of free PSA could help elucidate their prostatic origin and mechanism of release into the serum. However, attempts to purify and characterize the low levels of PSA from serum in the diagnostically relevant range near 10 ng/ml have not generally been considered feasible with current technologies. So far, studies have focused primarily on serum from men with unusually high levels of PSA, with 100s or 1000s of ng/ml PSA. We reported the presence of a truncated form of proPSA (PPSA) in pooled PCa serum containing 63 ng/ml total PSA (9). However, other groups have failed to identify pPSA in serum containing much higher levels of PSA. While studies using high serum PSA levels are suggestive, they suffer a common drawback in that this PSA may not reflect the kind or percentage of PSA that is typically present in the early stages of the disease, where PSA is 10 ng/ml or less. PSA released from large primary tumor lesions or metastatic disease may have different biochemical properties than PSA released from an early, possibly lower grade, disease. Therefore, in order to be useful as an aid in the clinical detection of early prostate cancer, the truncated pPSA forms would have to be present at significant levels in serum with diagnostically relevant levels of total PSA near 10 ng/ml.

When the levels of total PSA are near 10 ng/ml, men who have a higher proportion or percentage of free PSA are more likely to have a benign disease. Studies have characterized these levels of free PSA and have, for instance, recommended that men with greater than 25% of their total PSA present as free PSA not be biopsied for cancer since there is only an 8% probability of cancer in these cases. However, no known diagnostic methods are available to aid in the further detection of prostate cancer in the population of the men with 8% of cancer probability. In addition, when the total PSA is in the range from 2.5-4 ng/ml, many cancers are present but at a lower occurrence than when the total PSA is in the range from 4-10 ng/ml. Current attempts to use the ratio of free PSA to improve cancer detection in the 2.5-4 ng/mll range have yet to establish diagnostic value.

Therefore, a need exists to develop assays for improving the detection and determination of early prostate cancer in a patient.

### SUMMARY OF THE INVENTION

The present invention is based on the unexpected discovery that pPSA isoforms, for example, the [−2]pPSA form, have elevated cancer predictive value within specific subpopulations of men with a significantly elevated ratio of free PSA to total PSA, and particularly in men with greater than 25% free PSA. In addition, it is the discovery of the present invention that pPSA may also be effective in identifying a high percentage of men with prostate cancer when the total PSA in a sample of the patient is in the range from 2 to 4 ng/ml, a range where many cancers are present but at a lower occurrence. Thus, the present invention provides a means to aid in detection of prostate cancer in the very population of men where cancer is more rare, and where there is no current method to detect prostate cancer using immunoassays.

Accordingly, based on the discoveries of the present invention, one aspect of the present invention provides diagnostic methods to aid in detecting and/or determining the presence of prostate cancer in a subject, or to aid in distinguishing prostate cancer from BPH in a subject. In accordance with embodiments of the present invention, such a method includes the steps of:

a) determining the amount of total PSA contained in a biological sample from the patient;

b) determining the amount of free PSA in the sample; and calculating the ratio of the free PSA to the total PSA;

c) determining the amount of pPSA in the sample; and

d) correlating the amount of pPSA contained in the sample to the presence of prostate cancer in the patient by comparing the amount of pPSA to a predetermined value established with control samples of known cancer and benign disease diagnosis, based on both the level of total PSA and the % free PSA.

According to the embodiments of the present invention, the total PSA may be in the range of 2 to 10. The ratio of free PSA to total PSA may be greater than 10% to 25%. Alternatively, the total PSA may be in the range of 2.5 to 4 ng/ml, and the ratio of free PSA may be less than 15%. The pPSA may be [−2]pPSA, [−4]pPSA, or [−5/−7]pPSA. Particularly, the pPSA may be [−2]pPSA. The sample may be serum or plasma.

Kits for diagnosing or distinguishing prostate cancer from BPH are also included as embodiments of the present invention.

### BRIEF DESCRIPTION OF THE DRAWINGS

FIG. 1 shows the Receiver Operating Characteristic (ROC) analysis of [2,4,5,7]pPSA/ free PSA (%[2,4,5,7]pPSA, the sum of %[−2]+[−4]+[−5]+[−7]pPSA) compared to free PSA/total PSA (%F) in men with cancer and BPH and 4-10 ng/ml total serum PSA.

FIG. 2 shows the ROC analysis of [2]pPSA/free PSA (%[−2]pPSA) compared to free PSA/total PSA (%F) in men with cancer or BPH and 4-10 ng/ml total serum PSA.

FIG. 3 shows the ROC analysis of [−2]pPSA compared to free PSA/total PSA (%F) in men with cancer or BPH and 4-10 ng/ml total serum PSA.

FIG. 4 shows the ROC analysis of [−2]pPSA compared to free PSA/total PSA (%F) in men with cancer or BPH, 4-10 ng/ml total serum PSA and greater than 20% free PSA.

FIG. 5 shows the ROC analysis of [−2]pPSA compared to free PSA/total PSA (%F) in men with cancer or BPH, 4-10 ng/ml total serum PSA and greater than 25% free PSA.

FIG. 6 is the dot blot of the [−2]pPSA data from FIG. 5 showing the cutoff at 0.053 ng/ml.

FIG. 7 shows the ROC analysis of [2,4,5,7]pPSA/free PSA (%[2,4,5,7]pPSA, the sum of %[−2]+[−4]+[−5]+[−7]pPSA) compared to free PSA/total PSA (%F) in men with cancer and BPH and 2.5-4 ng/ml total serum PSA.

FIG. 8 shows the ROC analysis of [2,4,5,7]pPSA/free PSA (%[2,4,5,7]pPSA, the sum of %[−2]+[−4]+[−5]+[−7]pPSA) compared to free PSA/total PSA (%F) in men with cancer and BPH, 2.5-4 ng/ml total serum PSA and less than 15% free PSA.

### DETAILED DESCRIPTION OF THE PREFERRED EMBODIMENTS

One aspect of the present invention provides diagnostic methods to aid in detecting and/or determining the presence of prostate cancer in a subject, or to aid in distinguishing prostate cancer from BPH in a subject. In accordance with embodiments of the present invention, such a method includes the steps of:

a) determining the amount of total PSA contained in a biological sample from the patient;

b) determining the amount of free PSA in the sample; and calculating the ratio of the free PSA to the total PSA;

c) determining the amount of pPSA in the sample;

d) correlating the amount of pPSA contained in the sample to the presence of prostate cancer in the patient by comparing the amount of pPSA to a predetermined value established with control samples of known cancer and benign disease diagnosis, based on both the level of total PSA and the % free PSA.

Methods of measuring total PSA and free PSA are well known in the art, and therefore will not be repeated herein. For the purpose of the present invention, the term “total PSA ” refers to all immunologically available forms of PSA including free PSA and PSA complex with ACT. The term “free PSA” as used herein refers to PSA that is not complexed to any other protein but is found as the 30 kDa protein in solution.

As used herein, the terms “proPSA,” “pPSA,” “proPSA polypeptide”, and “pPSA polypeptide” are used interchangeably and preferably encompass all inactive precursor forms of PSA, including, but not limited to, [−2]proPSA, [−4]proPSA, [−7]proPSA, and [−5]proPSA.

The amount of pPSA may be measured by any methods described herein or known in the art, or later developed, as long as they are capable of making such a measurement. In accordance with one embodiment of the present invention, pPSA contained in a sample may be measured by a method including the steps of contacting an agent that specifically binds to proPSA with the sample under a condition that allows the formation of a binary complex comprising the proPSA and the agent, and detecting and determining the amount of the complex.

For the purpose of the present invention, an agent may be any molecular species capable of binding to pPSA with sufficient specificity. The binding specificity is sufficient if the binding allows the formation of a complex comprising the agent and pPSA, and the determination of the amount of the complex. Examples of potential molecular species include, but are not limited to, antibodies, antigen-binding fragments derived from antibodies, and equivalents of antibodies, such as, but not limited to, aptamers, etc. For the purpose of the present invention, an agent specific for pPSA may be selected by methods known in the art. For example, any known binding assays may be used to determine the specific binding activity of any given agent.

In accordance with embodiments of the present invention, proPSA may be measured by immunoassays. Antibodies and immunoassays that may be used for determining the amount of proPSA of the present invention are described in the co-pending U.S. applications Ser. Nos. 09/251,686, 09/302,965, 09,792,692; and 09,792,534, the content of which is incorporated herein in its entirety by reference. Specifically, the details of expressing PSA in mammalian cells, and making antibodies specific for PSA are provided in the co-pending U.S. patent applications, Ser. Nos. 09/251,686 and 09/302,965. As discussed in the co-pending application, a proPSA polypeptide or peptide corresponding to the proPSA region may be expressed and isolated from mammalian cells. Once isolated, the proPSA peptides may be used to produce anti-pPSA antibodies.

The details of how to make anti-pPSA antibodies are also described in the co-pending U.S. application Ser. No. 09,792,692. Briefly, the proPSA polypeptides used to generate antibodies in accordance with the invention include, but are not limited to, −7, −5, −4 and −2 proPSA. Peptides corresponding to the proPSA region can also be used to generate anti-pPSA antibodies and include all peptides which contain any portion of the pro region of the pPSA polypeptide. These peptides preferably contain about 8 to 15 amino acids and comprise an immunogenic epitope. In accordance with the embodiment of the invention, antibodies generated by the present invention preferably are specifically immunoreactive with, and bind to, proPSA of the present invention. The term “specifically immunoreactive or specifically binds to” as used herein indicates that the antibodies of the present invention recognize and bind to proPSA with less than 10% crossreactivity to other forms of PSA, such as other clipped or non-clipped mature forms of PSA. Examples of monoclonal antibodies that specifically bind to proPSA include, but are not limited to, PSIZ134, PSIZ120, PSIZ125, PSIZ80, PS2P206, PS2P309, PS2P446, PS2X094, PS2X373, PS2V411, and PS2V476. For example, monoclonal antibody PS2P446 is specific for [−7]/[−4]pPSA. PS2X373 is specific for [−2]pPSA. PS2V476 is specific for [−4]pPSA.

Antibodies of the present invention may be used for detecting and determining the presence and amount of proPSA in a sample. They may also be used for detecting and determining the presence and amount of different forms of proPSA in a sample. The detail description of immunoassays for detecting and determining the amount of proPSA or the different forms of proPSA is provided in co-pending U.S. patent application Ser. No. 09, 792,534, and will not be repeated herein.

A typical immunoassay of the present invention for determining the amount of any PSA of interest contained in a biological sample may include the steps of contacting an antibody that specifically binds to the PSA of interest with the sample under a condition that allows a formation of a binary complex comprising the PSA and the antibody; and (b) detecting and determining the amount of the complex. For the purpose of the present invention, any agents that are capable of forming a detectable complex with proPSA would be considered as equivalents of the antibody.

Typically, qualitative and/or quantitative determinations of any PSA of the present invention in a sample may be accomplished by competitive or non-competitive immunoassay procedures in either a direct or indirect format. Examples of such immunoassays are the radioimmunoassay (RIA) and the sandwich (immunometric) assay. The detection of the antigens using the monoclonal antibodies of the present invention can be done utilizing immunoassays which are run in either the forward, reverse, or simultaneous modes, including immunohistochemical assays on physiological samples. Those skilled in the art will know, or can readily discern, other immunoassay formats without undue experimentation.

The terms “immunometric assay” or “sandwich immunoassay” include a simultaneous sandwich, forward sandwich, and reverse sandwich immunoassay. These terms are well understood by those skilled in the art. Those skilled in the art will also appreciate that antibodies according to the present invention will be useful in other variations and forms of assays which are presently known or which may be developed in the future. These are intended to be included within the scope of the present invention.

For the purpose of the present invention, the biological sample can be any human physiological fluid sample that contains proPSA of the present invention. Examples of the human physiological fluid sample include, but are not limited to, serum, seminal fluid, urine, and plasma. In addition, both monoclonal antibodies and polyclonal antibodies may be used as long as such antibodies possess the requisite specificity for the antigen provided by the present invention. Preferably, monoclonal antibodies are used.

In accordance with the embodiment of the present invention, the immunoassays include assays specific for [−2]pPSA, [−4]pPSA and [−5/−7]pPSA. The specific forms of pPSA are measured in the serum of men with known prostate cancer or BPH, and the levels of these analytes are used to develop algorithms to distinguish prostate cancer from benign prostatic disease. The immunoassays of the present invention may be used to detect pPSA in human physiological samples, such as serum and tissue, for the purpose of aiding in diagnosing and monitoring prostate cancer. The assays of the present invention may also be used to aid in distinguishing prostate cancer from benign prostatic hyperplasia.

It is a surprise discovery of the present invention that pPSA, e.g., [−2]pPSA or the sum of [−2], [−4] and [−5/−7]pPSA, has a significantly elevated cancer predictive value when analyzing serum specimens containing 4-10 ng/ml total PSA and % free PSA greater than 20%, or particularly greater than 25%. In addition, in serum specimens with total PSA between 2.5 and 4 ng/ml, the pPSA shows increased cancer predictive value in those samples with less than 15% free PSA. Thus, the pPSA isoforms have a significantly elevated cancer predictive value within specific subpopulations of total PSA and % free PSA. The pPSA isoforms may also show increased cancer discrimination throughout the total range of % free PSA in a series of samples. This effect can be significantly enhanced when choosing selected % free PSA ranges or cutoffs. Examples described below show that the %[−2]pPSA above a certain cutoff is capable of detecting 11 out of 13 missed cancers in serum with greater than 25% free PSA.

Therefore, in accordance with embodiments of the present invention, the total PSA of a sample may be in the range of from about 2 to 10 ng/ml. The ratio of free PSA to total PSA in a sample may be greater than 10 to 25%, or less than 15%, when the amount of total PSA is in the range of 2 to 4 ng/ml. In a particular embodiment, the total PSA is in the range of 4 to 10 ng/ml, and the ratio of free PSA is in the range of greater than 20%, or 25%.

For the purpose of the present invention, the amount of pPSA detected in a sample of a patient may be correlated to the presence of prostate cancer in any way that generates diagnostic value for determining the presence of prostate cancer. In accordance with embodiments of the present invention, the amount of pPSA is compared to a predetermined value for determining the presence of prostate cancer. In view of the teaching of the present invention, one skilled in the art through routine experimentation can readily determine the “predetermined cutoff value” (or threshold) or other analytical parameters necessary to allow the use of the level of the pPSA in determining the presence of prostate cancer. For example, one may compare the above-discussed ratio of pPSA in individuals diagnosed with prostate cancer with individuals that do not have prostate cancer or have BPH to determine the cut-off values. Receiver Operating Characteristic (ROC) analysis can be used to determine a cutoff value with required specificity and sensitivity. ROC analysis is known in the art and is described in detail in reference 13 (13), and the relevant content of which is incorporated herein by reference. Then, the ratio or the amount of pPSA of a sample may be compared to the predetermined cut-off value for determining the presence of prostate cancer in a subject, wherein the higher level of pPSA may be an indication of prostate cancer. This and other methods of determining the cutoff value with required specificity and sensitivity are well known in the art and need not be repeated (14-21).

The invention is further described by reference to the following examples.

### EXAMPLE I

### Analysis of Serum of Men with Cancer and Benign Disease Containing Total PSA between 4 and 10 ng/ml

Material and Methods

Development of Monoclonal Antibodies (mAbs) to pPSA

mAbs to [−2] and [−4]pPSA were developed by mouse immunization with peptides attached to Keyhole limpet hemocyanin (Pierce Chemical Co. Rockford, Ill). For [−2]pPSA, the pro peptide was SRIVGGWECEK, and for [−4]pPSA, the peptide was ILSRIVGGWECEK. Hybridomas were produced by usual methodologies19 and the antibody clones selected by reactivity to the respective peptide indicated above, and no reactivity to the control peptide for mature PSA, IVGGWECEK. The clones were further selected on their ability to recognize purified [−2] and [−4]pPSA protein on Western blots. When SDS-PAGE was run under reducing conditions, PS2X373 showed about 20% cross-reactivity to the mature PSA protein by Western blot. However, the cross-reactivity dropped to 5% or less under non-reducing conditions and so these conditions were used for the detection of [−2]pPSA in the Results. None of the clones generated using peptide immunogens were capable of recognizing native [−2] or [−4]pPSA proteins in solution by standard immunoassay techniques in microtiter plates.

mAbs to full length [−7]pPSA were obtained by mouse immunization with purified recombinant chimeric protein consisting of the PSA prepro leader peptide attached to human kallikrein 216,20. Clones were screened on their recognition of native recombinant pPSA, and no recognition of native mature PSA. These mAbs were found to recognize both the [−7]pPSAa and the [−5]pPSA by immunoassay, and therefore these antibodies in assay format may alternately be referred to as an assay for [−7]pPSA or [−5/−7]pPSA.

Isolation of Recombinant pPSA from Mammalian Cells

Recombinant PSA was expressed in mammalian AV12 cells as described earlier. The spent media was passed over the PSA-specific mAb, PSM773. PSM773 has been shown previously to have specificity for mature, clipped, and precursor forms of PSA (10-12). The column was washed with 40 volumes of PBS containing 0.1% reduced Triton-X100, and bound protein eluted with 100 mM glycine pH 2.5, containing 200 mM sodium chloride. The eluant was immediately neutralized with 10% % vol/vol IM Tris pH 8.0. The purified PSA contained no mature PSA but contained [−5/−7], [−4], and [−2]pPSA molecular isoforms of pPSA that were purified by HIC-HPLC (12).

Immunoassay of PSA

The concentration of PSA in serum and purified preparations was determined by Tandem®-MP PSA and Tandem®-MP free PSA assays (Hybritech Incorporated, San Diego, Calif.; Beckman Coulter, Inc., Fullerton, Calif.).

Immunoassay of [−2], [−4] and [−5/−7]pPSA

The immunoassay we have developed for the measurement of the pPSA forms is as follows. 50 ul of biotinylated anti-PSA Ab PSM 773 at 5 ug/ml in Tandem® PSA zero cal diluent is added to a EG&G Wallac streptavidin coated microplate and allowed to react at room temperature for 1 hour with shaking. The plate is then washed 5 times with Tandem® E wash. 50 ul of Tandem® PSA zero cal diluent is then added to the plate followed by 50 ul of sera or antigen to be tested. The mixture is allowed to react at room temperature for 2 hours as above. The plate is then washed 5 times with Tandem® E wash. 100 ul of a ImA solution of the appropriate Europeum-labelled detect mAb for the intended measurement of each form of pPSA is added to the plate. For [−2]pPSA, this is PS2X373; for [−4]pPSA, PS2V 411; and for [−5/−7]pPSA, this is PS2P309. The mixture is allowed to react at room temperature for 1 hour as above. The plate is then washed 5 times with Tandem® E wash and read on a EG&G Wallac Victor instrument.

Receiver Operating Characteristic (ROC) Curve Analysis

ROC analysis and graphs were generated using MedCalc software program (MedCalc Software, Belgium, (info@medcalc.be)). Theory and practice is described in: Zwieg and Campbell, Receiver-operating characteristic (ROC) : a fundamental evaluation tool in clinical medicine, Clinical Chemistry, 39, 561-577, 1993.

### Results

The total PSA, free PSA and [−2]pPSA, [−4]pPSA and [−5/−7]pPSA immunoassay measurements were performed on 303 serum samples which contained a known diagnosis of 107 cancer and 196 benign disease. Values of each component were calculated and expressed as ng/ml of serum. The serum values for each PSA isoform were entered into the MedCalc program in order to generate an ROC curve analysis. ROC is a statistical method to assign a positive and negative predictive value of cancer for each sample in the cohort and is the most widely used method to assess the value and performance of PSA and free PSA assays as predictors of cancer. In the simplest of terms, the area under the curve, AUC, for each ROC analysis is used to assess assay predictive value. A higher AUC value indicates a better overall predictive value. ROC values range from 0.5 (no improvement over random chance) to 1.0 (a perfect assay with 100% predictive value).

In most cases, the objective of the ROC analysis is to maximize the discrimination of men with cancer and to minimize biopsies on those men who do not have cancer, i.e., false positives. When analyzing biological samples such as PSA in serum, there is a balance between the detection of true positives and false positives. For example, depending on the desired outcome, it may be most desirable to detect 95% of the cancers, and this is inevitably accompanied by a high false positive rate for those men with the benign disease. In the case of % free PSA, for example, in order to detect 95% of the cancers, it requires biopsies of all men who have less than 25% free PSA. This is, however, accompanied by an 80% false positive rate. Under different circumstances, it may be most desirable to minimize the false positive rate, in which case only a small percentage of the true cancers would be detected. This second paradigm might be desirable when dealing with a large population with a relatively small occurrence of cancer. It might not be feasible to biopsy the entire population, and so one establishes a balance and detects as many cancers as is feasible for a given number of biopsies. These hypothetical examples are meant to demonstrate that the assessment and value of the ROC analysis is not absolute, and in the case of the current invention with pPSA, will require experiments to establish the exact parameters to be used in practice. However, in view of the teaching of the present invention, one skilled in the art should be able to readily determine the necessary parameters without undue experimentation. Examples given below are meant to demonstrate the value and efficacy of pPSA in detecting cancer in a given population, and under a given set of criteria, but the uses and cutoffs for pPSA are not limited to these examples.

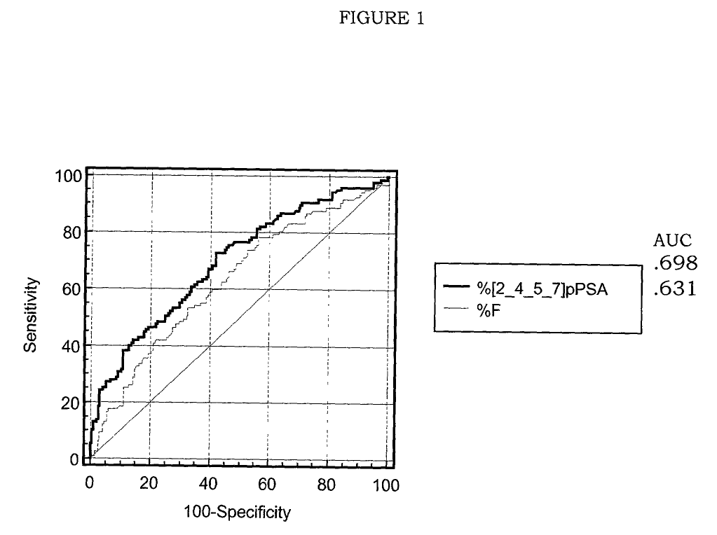

In the field of PSA testing, it is known that the % free PSA improves cancer detection compared to total PSA alone in the early cancer detection range of 4-10 ng/ml total PSA. In the sample set shown in FIG. 1, the % free PSA also had a higher predictive value than PSA and so the pPSA measurements in these samples were evaluated for their ability to improve cancer predictive value compared to the % free PSA. FIG. 1 shows the comparison of the %F (the % free PSA, or free/total PSA) and the %[2,4,5,7]pPSA (the sum of all the pPSA forms divided by the free PSA, i.e., the percent of the free PSA that is composed of all pPSA forms). In this case, the %[2,4,5,7]pPSA shows a mild improvement over the %F as seen by the higher AUC of 0.698 compared to 0.631 for %F.

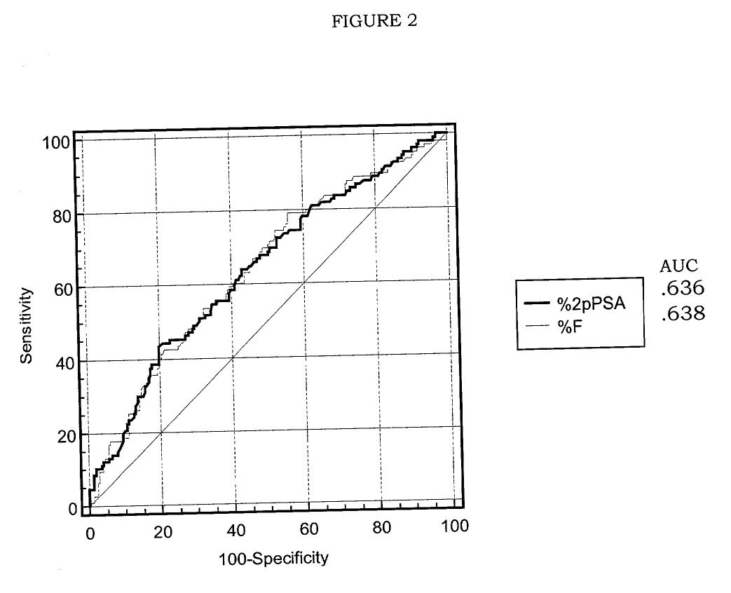

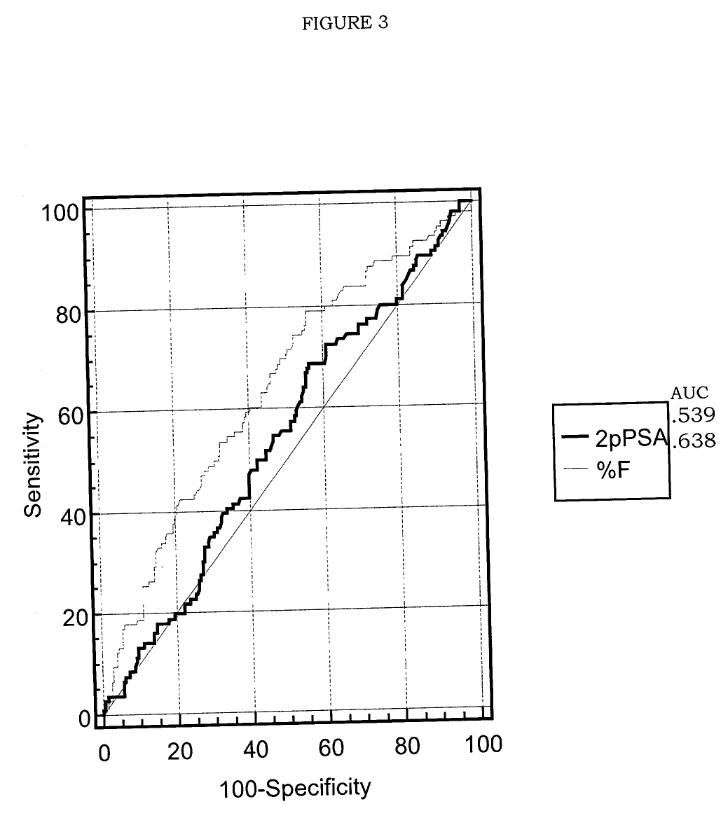

FIG. 2 uses a slightly different paradigm and compares the individual pPSA component %[−2]pPSA to %F. The AUC for this comparison indicates that both analytes have almost equal ability to predict cancer. FIG. 3 shows the comparison of [−2]pPSA to %F. In this case, the [−2]pPSA represents the ng/ml of this pro form in the serum, and not as a % of the free PSA. The AUC analysis indicates that [−2]pPSA by itself has less ability to predict cancer than % free PSA.

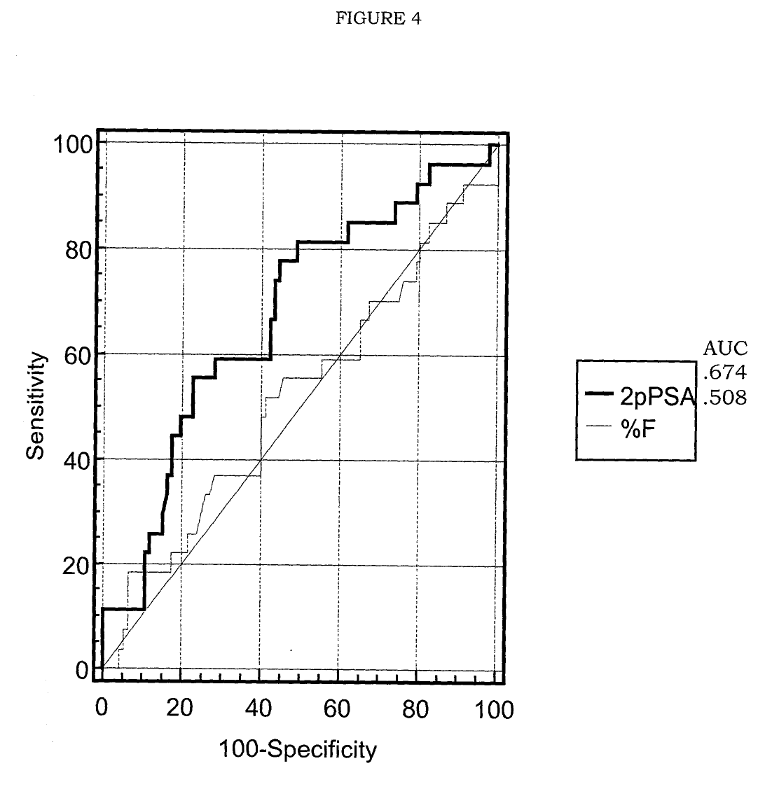

However, in FIG. 4, a subset of the whole sample population of 303 was analyzed. Only those men with greater than 20% free PSA were selected for ROC analysis. This represented 120 of the 303 samples. Whereas the entire population contained 35% cancer, this 120 sample subgroup contained only 23% cancer, consistent with the overall trend that the relative percentage of cancer decreases with increasing %F. In FIG. 4, in men with greater than 20% free PSA, the measurement of %F has virtually no ability to predict cancer, with an AUC of 0.504. By contrast, within this same range, the [−2]pPSA shows a significantly enhanced ability to predict cancer as indicated by an AUC of 0.674. Thus, [−2]pPSA shows a much higher specificity for cancer detection in a population with elevated % free PSA, where cancers are fewer and more difficult to detect.

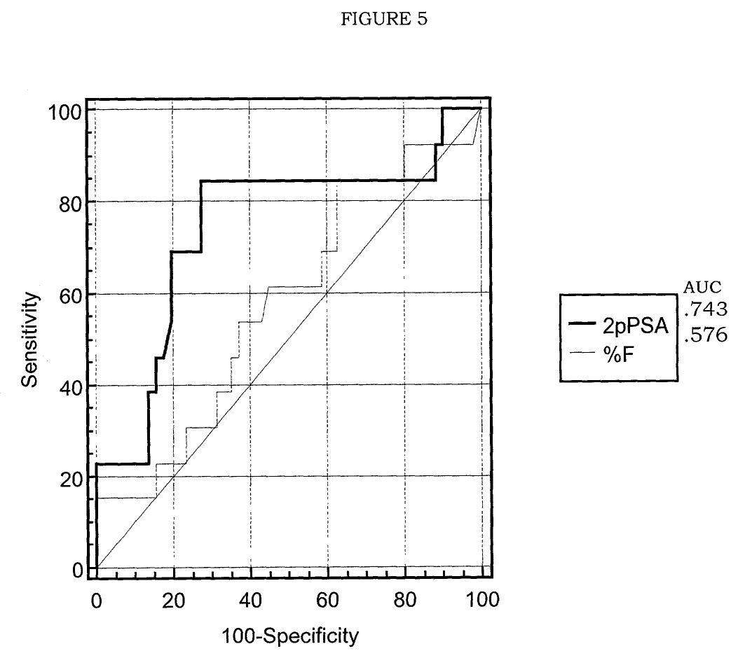

A more dramatic and practical application of this phenomenon is seen in FIG. 5. In this figure, only men with % free PSA greater than 25% were analyzed. In truly random screening populations, it is recommended that men with greater than 25% free PSA not be biopsied for cancer since the probability is only about 8% that cancer will be found. Thus, cancers in this group of men would tend to remain undetected under normal circumstances since there is no routine blood test to predict cancer in this group. FIG. 5 shows that [−2]pPSA has a highly enhanced predictive value for cancer in this group, with an AUC of 0.743. The fact that the AUC is even higher for [−2]pPSA at greater that 25% free PSA than in the greater than 20% free PSA cutoff confirms the overall observation that [−2]pPSA becomes a better predictor of cancer as the % free PSA rises. It was unanticipated that the pPSA forms would show selective cancer prediction in a specific range or cutoff of % free PSA.

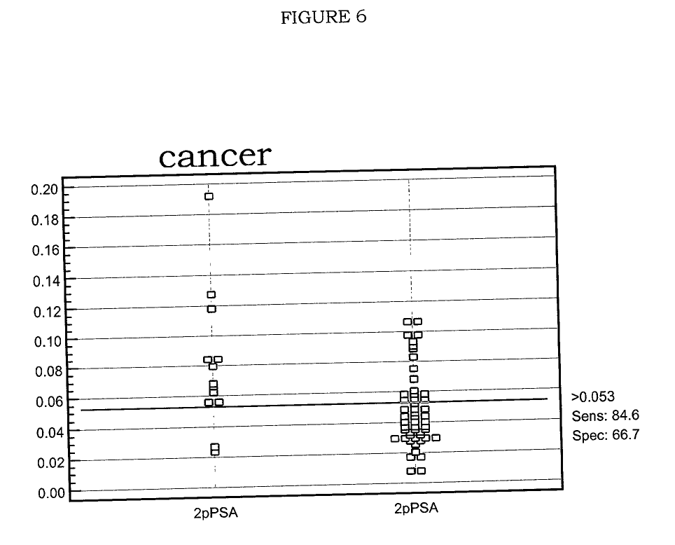

A practical example of how the pPSA assay could be used is seen in FIG. 6, which shows the dot-plot for the individual values in FIG. 5, plotted from the same program that generated FIG. 5. In the population with greater than 25% free PSA, there are 13 cancers and 51 non-cancers, assumed to be primarily BPH. Using a cutoff of 0.053 ng/ml [−2]pPSA, one would detect 11 of the 13 cancers (85% sensitivity), while biopsying only 17 of the 51 (67% specificity) non-cancers in this group. Again, this is given as an example of how pPSA might be used to detect prostate cancer, but does not imply that 0.053 ng/ml [−2]pPSA will be the ultimate cutoff used for these types of samples. More extensive analysis with larger control populations with known cancer and BPH will be useful for determining an appropriate cutoff value. And as stated earlier, this is not the only approach that may be desirable with different populations of patients.

Though not shown for the greater than 25% free PSA samples, other pPSA metrics such as %[−2]pPSA, [−2]pPSA/total PSA, %[2,4,5,7]pPSA and others showed improved ROC AUCs compared to %F.

### EXAMPLE II

### Analysis of Serum of Men with Cancer and Benign Disease Containing Total PSA between 2.5 and 4 ng/ml

As in Example 1, the same series of PSA assays were measured in this cohort of patients who have total PSA values from 2.5-4 ng/ml. There were 286 total samples with 109 cancers and 177 benign disease samples. The range from 2.5-4 ng/ml is an area where many cancers are present but at a lower occurrence than in the 4-10 ng/ml patients in random screening populations. Current attempts to use %F to improve cancer detection in this range have yet to establish diagnostic value.

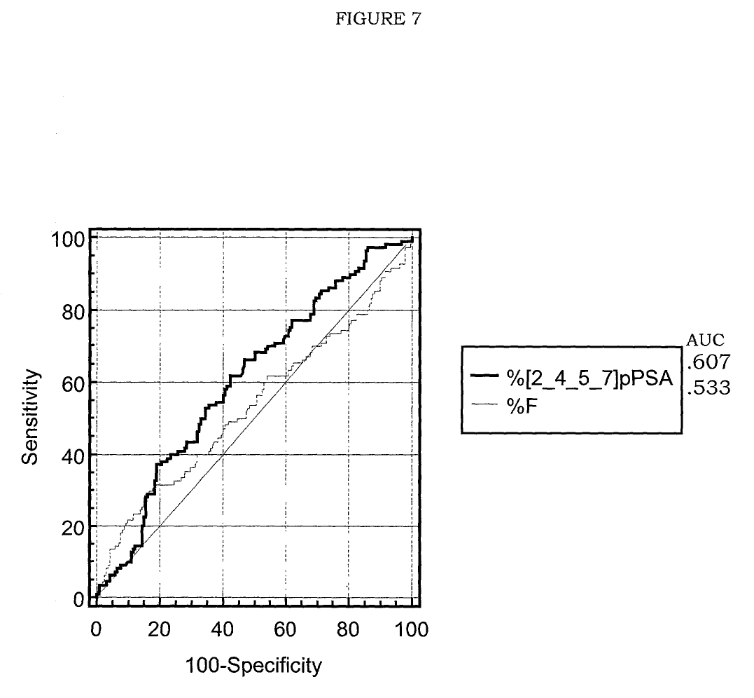

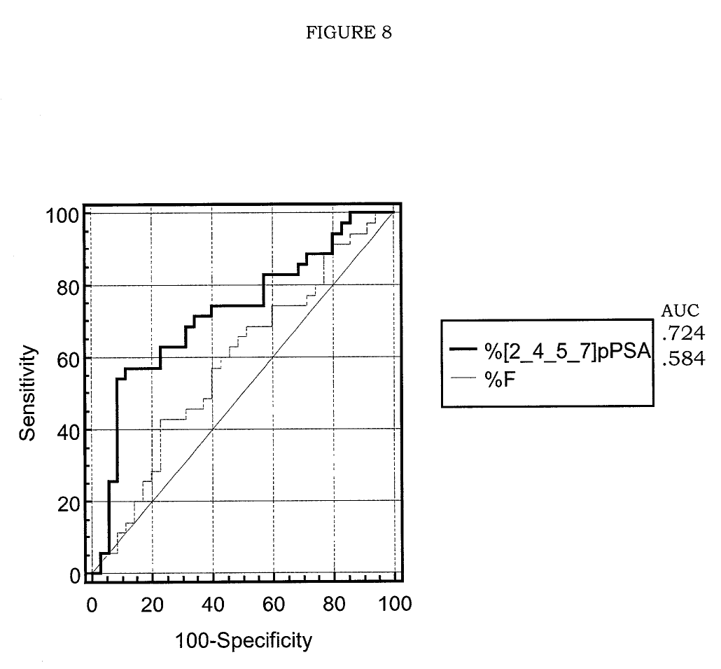

FIG. 7 shows an ROC comparison of %[2,4,5,7]pPSA to %F. The %[2,4,5,7]pPSA shows significantly improved predictive value for cancer. In FIG. 8, only samples with less than 15% free PSA were subjected to an ROC analysis. The %[2,4,5,7]pPSA showed an even more pronounced increase in AUC compared to %F, indicating an improved cancer predictive value. This is one example of a case where there is a large population with a low probability of cancer and it may be most desirable in this case to minimize the false positives by setting the parameters to select only those with a very high probability of cancer, i.e., high specificity.

### Discussion

These examples demonstrate that the pPSA isoforms of free PSA act as independent markers for cancer detection compared to free PSA as a whole. This is true throughout the current diagnostic PSA range of interest, 2.5-10 ng/ml. The sum of all pPSA isoforms, or individual isoforms of pPSA, improve cancer detection in patient populations, as earlier proposed in application Ser. No. 09/302,965, filed on Apr. 30, 1999 (which in turn is a continuation-in-part of application Ser. No. 09/251,686, filed on F Feb. 17, 1999, which in turn is a continuation of application Ser. No. 08/846,408, filed on Apr. 30, 1997).

However, it was unexpected that dramatically increased predictive values would be so pronounced in samples stratified by their %F. This is a novel finding and suggests a complex relationship between pPSA and the total percent of free PSA in the serum. The measurement of pPSA can be applied throughout the early diagnostic range of 2-10 ng/ml PSA in order to improve cancer detection and reduce unnecessary biopsies. The examples given in the Results are meant to demonstrate applicability of pPSA isoforms towards prostate cancer detection, and to suggest some relevant examples of such, but these examples are not meant to limit other potential applications with different ranges of PSA, % free PSA or pPSA cutoffs.

### REFERENCES LIST:

1. Catalona, W. J., Smith, D. S., Ratliff, T. L., Dodds, K. M., Coplen, D. E., Yuan, J. J., Tetros, J. A., and Andriole, G. L. Measurement of prostate-specific antigen in serum as a screening test for prostate cancer. N. Engl. J. Med., 324:1156-1161, 1991.

2. Oesterling, J. E. Prostate-specific antigen: a critical assessment of the most useful tumor marker for adenocarcinoma of the prostate. J. Urol., 145:907-923, 1991.

3. Labrie, F., Dupont, A., Suburu, R., Cusan, L., Tremblay, M., Gomez, J. L., and Emond, J. Serum prostate specific antigen as pre-screening test for prostate cancer. J. Urol., 147:846-851, 1992.

4. Lilja, H., Christensson, A., Dahlen, U., Matikainen, M. T., Nilsson, O., Pettersson, K., and Lovgren, T. Prostate-Specific Antigen in Serum Occurs Predominantly in Complex with α₁-antichymotrypsin. Clin. Chem, 37:1618-1625, 1991.

5. Stenman, U. H., Leinonen, J., Alfthan, H., Rannikko, S., Tuhkanen, K., and Alfthan, O. A complex between prostate-specific antigen and α₁antichymotrypsin is the major form of prostate-specific antigen in serum of patients with prostatic cancer: assay of the complex improves clinical sensitivity for cancer. Cancer Res., 51:222-226, 1991.

6. Catalona, W. J., Partin, A. W., Slawin, K. M., Brawer, M. K., Flanigan, R. C., Patel, A., Richie, J. P., deKemion, J. B., Walsh, P. C., Scardino, P. T., Lange, P. H., Subong, E. N., Parson, R. E., Gasior, G. H., Loveland, K. G., and Southwick, P. C. Use of the percentage of free prostate-specific antigen to enhance differentiation of prostate cancer from benign prostatic disease: a prospective multicenter clinical trial. JAMA, 279:1542-1547, 1998.

7. Woodrum, D. L., Brawer, M. K., Partin, A. W., Catalona, W. J., and Southwick, P. C. Interpretation of free prostate-specific antigen clinical research studies for the detection of prostate cancer. J. Urol., 159:5-12, 1998.

8. Peter, J., Unverzagt, C., and Hoesel, W. Analysis of free prostate-specific antigen (PSA) after chemical release from the complex with alpha(1)-antichymotrypsin (PSA-ACT). Clin. Chem., 46:474-482, 2000.

9. Mikolajczyk, S. D., Grauer, L. S., Millar, L. S., Hill, T. M., Kumar, A., Rittenhouse, H. G., Wolfert, R. L., and Saedi, M. S. A precursor form of PSA (PPSA) is a component of the free PSA in prostate cancer serum. Urology, 50:710-714, 1997.

10. Wang, T. J., Linton, H. J., Sokoloff, R. L., Grauer, L. S., Rittenhouse, H. G., and Wolfert, R. L. Antibody specificities for PSA and PSA fragments by SDS-PAGE Western blot analysis. Tumor Biology, 20:7578, 1997.

11. Finlay, J. A., Day, J. R., and Rittenhouse, H. G. Polyclonal and Monoclonal Antibodies to Prostate-Specific Antigen can Cross-React with Human Kallikrein 2 and Human Kallikrein 1. Urology, 53:746-751, 1999.

12. Kumar, A., Mikolajczyk, S. D., Goel, A. S., Millar, L. S., and Saedi, M. S. Expression of pro form of prostate-specific antigen by mammalian cells and its conversion to mature, active form by human kallikrein 2. Cancer Res., 57:3111-3114, 1997.

13. Dawson, J. M. 1999. Clinical Trials: Analysis and Presentation, Presented at the 26^{th} Annual Meeting of the Association of Medical Diagnostics Manufacturers, Available at http://www.amdm.org/AMDM/NewsletterV12-2-2.html

14. Linnet, K. 1999. Necessary sample size for method comparison studies based on regression analysis. Clin. Chem., 45: 882-94.

15. Linnet, K. 1993. Evaluation of regression procedures for methods comparison studies. Clin. Chem., 39: 424-32.

16. Bland, J. M., Altman, D. G. 1986. Statistical methods for assessing agreement between two methods of clinical measurement. Lancet, i: 307-10.

17. Reid, M. C., Lachs, M. S., Feinstein A. R. 1995. Use of methodologic standards in diagnostic test research. Getting better but still not good. JAMA, 274: 645-51.

18. Krouwer, J. S. Cumulative distribution analysis graphs—an alternative to ROC curves [Tech Brief]. Clin. Chem., 33: 2305-6.

19. Albert, A. 1982. On the use and computation of likelihood ratios in clinical chemistry. Clin. Chem., 28: 1113-9.

20. Solberg, H. E. 1978. Discriminant analysis. Crit. Rev. Clin. Lab. Sci., 9: 209-42.

21. Matthews, J. N. S., Altman, D. G., Campbell, M. J., Royston, P. 1990. Analysis of serial measurements in medical research. Br. Med. J., 300: 230-5.

We claim:

1. A method to aid in detecting or determining the presence of prostate cancer in a patient, comprising the steps of:
a) determining the amount of total PSA contained in a biological sample from the patient;
b) determining the amount of free PSA in the sample; and calculating the ratio of the free PSA to the total PSA;
c) determining the amount of pPSA in the sample; and
d) correlating the amount of pPSA contained in the sample to the presence of prostate cancer in the patient by comparing the amount of pPSA to a pre-determined value established with control samples of known cancer and benign disease diagnosis, based on both the level of total PSA and the % free PSA.

2. The method of claim 1, wherein the sample is serum or plasma.

3. The method of claim 1, wherein the total PSA is between 2.5 and 10 ng/ml, the ratio of free PSA to total PSA is greater than 20%, and the amount of the pPSA that is above the pre-determined value is an indication of the presence of prostate cancer.

4. The method of claim 3, wherein the total PSA is between 4 to 10 ng/ml.

5. The method of claim 4, wherein the ratio of free PSA to total PSA is greater than 25%.

6. The method of claim 1, wherein the total PSA is between 2.5 and 10 ng/ml, the ratio of free PSA to total PSA is selected from percentages between 5% and 25%, and the amount of the pPSA that is above the pre-determined value is an indication of the presence of prostate cancer.

7. The method of claim 6, wherein the total PSA is between 4 to 10 ng/ml.

8. The method of claim 1, wherein the pPSA is selected from a group consisting of [−2]pPSA, [−4]pPSA, [−5]pPSA, and [−7]pPSA.

9. The method of claim 1, wherein the pPSA is any combination selected from a group consisting of [−2]pPSA, [−4]pPSA, [−5]pPSA, and [−7]pPSA.

10. The method of claim 8, wherein the pPSA is [−2]pPSA.

11. The method of claim 1, wherein the total serum PSA is between 2.5 and 4 ng/ml.

12. The method of claim 11, wherein the ratio of free PSA to total PSA is selected from percentages between 5% and 25%.

12. The method of claim 11, wherein the ratio of free PSA to total PSA is less than 15%.

13. The method of claim 1, wherein the total PSA is selected from any combination of increments from 2.0 to 10.0 ng/ml.
